(19) United States
(12) Patent Application Publication
(10) Pub. No.: US 2009/0047341 A1
(43) Pub. Date: Feb. 19, 2009

(54) CAPSULES OF CORE/SHELL TYPE AND PREPARATION PROCESS

(75) Inventors: Jean-Thierry SIMMONET, Cachan (FR); Bruno BIATRY, Vincennes (FR); Fanny PRIGENT, Paris (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 12/166,024

(22) Filed: Jul. 1, 2008

Related U.S. Application Data
(60) Provisional application No. 60/970,000, filed on Sep. 5, 2007.

(30) Foreign Application Priority Data
Jul. 5, 2007 (FR) 07 56298

(51) Int. Cl. A61K 9/66; A61Q 17/04; A61P 17/00

(52) U.S. Cl. 424/455; 424/59

(57) ABSTRACT

The present invention relates to a process for the preparation of a suspension of capsules of core/shell type comprising the steps of mixing, at ambient temperature, an oily phase and polymers, at least one of which is an oligomer having a weight-average molecular weight of less than 10 000 and a hydroxyl number (N_{OH}) of greater than or equal to 10 mg KOH/g, of homogenizing the oily phase and the polymers by heating, of dispersing the homogeneous mixture thus obtained in an aqueous phase, in order to obtain a direct preemulsion, the oily phase of which comprises the polymers, of subjecting the preemulsion thus obtained to shear forces appropriate for the reduction in the diameter of the particles of the dispersed mixture, of cooling the emulsion thus obtained to a temperature suitable for the coacervation of the said polymers and for the coating of drops of the said oily phase by the coacervates, and of cooling the suspension to a temperature suitable for the formation of the capsules by the precipitation and/or crystallization of the coacervates. The present invention also relates to the capsules thus obtained.

This non provisional application claims the benefit of French Application No. 07 56298 filed on Jul. 5, 2007 and U.S. Provisional Application No. 60/970,000 filed on Sep. 5, 2007.

A subject-matter of the present invention is a novel process for the preparation of a suspension of capsules, uncoated or coated with a lamellar phase, of core/shell type comprising a lipid core forming or comprising a lipophilic active principle and a continuous water-insoluble shell (or casing) comprising at least one crosslinked or noncrosslinked polymer, in particular an oligomer, with a molecular weight of less than 10 000 which is immiscible with water and in the lipid core.

The present invention also relates to the capsules and to a suspension of capsules which are capable of being obtained according to the process of the invention and also to the compositions comprising such a suspension and to their applications in the cosmetic, dermatological or pharmaceutical fields.

The encapsulation or the absorption of active agents in capsules of submicronic dimensions (of less than 1 μm) is known and used in particular in the cosmetic and dermatological fields. This is because these capsules, known as nanocapsules, are capable of passing through the surface layers of the stratum corneum and of penetrating into the upper layers of the living epidermis in order to release the active principle therein. This penetration into deeper layers of the epidermis broadens the area of action of the active agents and shelters them from rapid removal by simple rubbing.

Various techniques for the encapsulation of active agents, in particular lipophilic active agents, are known to a person skilled in the art.

EP 0 274 961 (or U.S. Pat. No. 5,049,322), EP 0 447 318 (or U.S. Pat. No. 6,203,802) or EP 1 029 587 (or U.S. Pat. No. 6,565,886) describe a process for the encapsulation of oily or fat-soluble active agents using a water-miscible organic solvent. However, this process exhibits the disadvantage of imposing a stage of evaporation of the solvents and, if appropriate, of a fraction of the water in order to increase the degree of encapsulation of the active agents.

What is more, the degree of encapsulation remains relatively low, generally not exceeding 8% and more generally 5% by weight, with respect to the weight of the dispersion.

FR 2 864 900 (or US 2005-175651) describes an encapsulation process which makes possible a degree of encapsulation of 10 to 15% but which employs a water-immiscible organic solvent, such as dichloromethane.

Such organic solvents can be the cause of safety and/or toxicity and/or ecotoxicity problems.

EP 1 462 157 describes microcapsules varying from 100 nm to 5 mm obtained by a complex coacervation process resulting from the interaction of two polymers, one cationic and the other anionic.

U.S. Pat. No. 4,124,526 describes particles obtained by coacervation of a salt of a polycarboxylic polymer by acidification of a suspension to pH 5 to 8. This process exhibits the disadvantage of resulting in particles being obtained which are heterogeneous in size and which have a size generally of greater than one μm and also of resulting in a high porosity.

WO 01/52848 (or U.S. Pat. No. 6,451,345) describes microcapsules, the size of which varies from 100 to 500 μm, obtained by coacervation of a polymer of ethylcellulose type in cyclohexane. This process exhibits the disadvantage of requiring the addition of a polymer, such as polyethylene, in order to bring about the coacervation and subsequently of evaporating the solvent.

WO 99/43426 (or US 2003-180235) describes lipid particles which are solid between 25 and 85° C., which vary from 10 nm to 5 mm in size, which are composed of at least one lipid of wax type and which are devoid of a solid polymeric coating. These particles exhibit the disadvantage of exhibiting a limited stability in combination with surface-active agents.

It is an object of the present invention to overcome the abovementioned disadvantages.

Another object of the present invention is to provide a suspension of capsules exhibiting an improved degree of encapsulation of active agents.

Another object of the present invention is to provide a process for the preparation of a suspension of capsules which is devoid of a stage of evaporation of water or of solvent, in particular of organic solvent.

Another object of the present invention is to provide a process for the preparation of a suspension of capsules which makes it possible to dispense with the use of organic solvent.

Another object of the present invention is to provide a process for the preparation of a suspension of capsules which makes it possible to obtain capsules which are small in size, in particular of the size of less than one tenth of a micron, indeed even less than a micrometre, and which exhibit a homogeneous distribution in size.

Another object of the present invention is to provide a process for the preparation of a suspension of capsules which makes it possible to obtain capsules having improved leaktightness.

A further object of the present invention is to provide capsules devoid of or exhibiting a very low density of pores at their surface. These pores are frequently observed in capsules prepared according to processes comprising a stage of evaporation of solvent. Such capsules advantageously exhibit improved leaktightness.

Thus, another object of the present invention is to provide core/shell capsules exhibiting improved leaktightness, in particular from the viewpoint of encapsulated fat-soluble active agents.

Thus, according to one of its aspects, a subject-matter of the present invention is a process for the preparation of a suspension of capsules of core/shell type comprising at least the steps of:

a) having a heterogeneous mixture at ambient temperature (T_{amb}) comprising:
an oily phase,
identical or different polymers which are immiscible with water and with the oily phase, at least one polymer of which is an oligomer having a weight-average molecular weight of less than 10 000 and a hydroxyl number (N_{OH}) of greater than or equal to 10 mg KOH/g,

b) homogenizing the oily phase and the polymers by heating the mixture obtained in step a) at a homogenization temperature TH, TH being less than 100° C., the said mixture having, at the temperature TH, a viscosity of less than or equal to 17 mPa·s,

c) dispersing, at the temperature TH, the homogeneous mixture obtained in step b) in an aqueous phase, in order to obtain a direct preemulsion, the oily phase of which comprises the polymers,

d) subjecting the preemulsion obtained in step c) to a combination of shear forces appropriate for the reduction in the diameter of the particles of the dispersed mixture to a mean size of less than or equal to approximately 50 μm,

e) cooling the emulsion obtained in step d) to a temperature Tc suitable for the coacervation of the said polymers and for the coating of drops of the said oily phase by the coacervates, and

f) cooling the suspension obtained in step e) to a temperature Tp suitable for the formation of the expected capsules by the precipitation and/or crystallization of the coacervates,

the oily phase being suitable for the formation of a homogeneous mixture with the said polymers, at a temperature T_{H}, and for the appearance of a phenomenon of coacervation of the said polymers at a temperature T_{c}.

The inventors have observed, unexpectedly, that the use of polymers comprising at least one or several oligomer(s) with a molecular weight of less than 10 000 and with a hydroxyl number (N_{OH}) of greater than or equal to 10 mg KOH/g which are immiscible with water and with the oily phase, with an oily phase capable of dissolving at least 10% of these polymers by heating, makes it possible to obtain mixtures capable of undergoing a phenomenon of coacervation resulting, after curing of the coacervates, in capsules of core/shell type being obtained which are small in size and homogeneous in size distribution and which have a core formed of the oily phase.

In particular, the inventors have shown, for example, that polycaprolactone oligomers (Capa®2201 or Capa® HC 1100 from Solvay) with shea butter and/or isopropyl N-lauroylsarcosinate, heated to 80° C., make it possible to obtain capsules with the properties required by the present invention.

The conditions for producing the coacervates advantageously make it possible to avoid the use of organic solvent(s) in a process of the invention.

A process of the invention can advantageously make it possible to encapsulate large amounts of active agent(s).

Furthermore, the preparation of the coacervates of the invention can also make it possible to adsorb and/or absorb large amounts of active agent(s).

A process according to the invention can optionally comprise an additional step g) of crosslinking of the oligomers.

The capsules of the invention may or may not be coated with a lamellar phase.

The capsules of the invention exhibit a better stability, an improved degree of encapsulation, a homogeneous distribution in size and a mean diameter in size of less than or equal to 50 μm, in particular of less than or equal to 10 μm and especially of less than or equal to 1 μm.

Within the meaning of the invention, “T_{amb}” is understood to mean a temperature varying from 20 to 25° C.

Another subject-matter of the present invention is capsules capable of being obtained by means of a process of the invention.

Thus, another subject-matter of the present invention is capsules of core/shell type composed:
of a core comprising an oily phase, and
of a continuous shell which is insoluble in water and in the oily phase and which is obtained by coacervation of identical or different polymers which are immiscible with water and with the oily phase, at least one polymer of which is an oligomer having a weight-average molecular weight of less than 10 000.

The oily phase may comprise at least one fat-soluble active agent and/or at least one fat-dispersible active agent or may itself form an active agent.

The capsules of the invention are generally obtained in an aqueous suspension, as is shown by the preparation process described above. Consequently, another subject-matter of the invention is an aqueous suspension of capsules of core/shell type such as are described above.

Another subject-matter of the present invention is thus a suspension of capsules which is capable of being obtained by means of a process of the invention.

The capsules according to the invention and the aqueous suspensions comprising them may, for example, be used in the preparation of cosmetic, dermatological or pharmaceutical compositions, for example for topical application.

Another subject-matter of the present invention is a process for the preparation of a cosmetic, dermatological or pharmaceutical composition comprising at least the step of mixing, with a physiologically acceptable medium, a suspension of capsules which is obtained according to the process according to the invention.

Another subject-matter of the invention is thus a cosmetic, dermatological or pharmaceutical composition comprising, in a physiologically acceptable medium, one or more capsules as described above or a suspension of capsules such as described above.

The term “physiologically acceptable medium” is understood to mean a medium compatible with use by a living being, for example a human being or an animal. In particular, such a medium may be appropriate for application on keratinous substances, for example the skin, mucous membranes, nails, scalp and hair.

Capsules of Core/Shell Type

Within the meaning of the invention, the term “capsule of core/shell type” is understood to mean particles having a structure composed of a lipid nucleus (or core), if appropriate forming or comprising an active agent, which nucleus is encapsulated in a continuous protective casing (or shell) which is insolvent in water and in the lipid nucleus.

In other words, they are, according to the invention, capsules with a liquid or semiliquid lipid core surrounded by a shell of polymeric nature and more particularly oligomeric nature.

These particles are consequently distinct from particles of sphere type composed of a porous polymeric matrix in which the active agent is absorbed and/or adsorbed.

The capsules of the invention advantageously have a homogeneous particle size distribution.

The particle size distribution describes the distribution of the size of the capsules around a mean size. The particle size distribution may be characterized by a mean diameter in size and a polydispersity index or a uniformity coefficient. The lower the value of the index or coefficient, the more homogeneously the sizes of the capsules are distributed around a mean size.

The capsules of the invention may have a mean diameter in size of less than or equal to 50 μm, in particular of less than or equal to 10 μm, in particular varying from 50 nm to 10 μm, in particular varying from 100 to 300 nm and more particularly varying from 150 to 200 nm.

For the capsules with a mean size of less than 1 μm (submicronic), the particle size distribution can be characterized by a polydispersity index, recorded as PI (dimensionless value characterizing the range of the particle size distribution), and the size can be expressed as mean size by intensity supplied by a Brookhaven type BI90PLUS® particle sizer, the measurement principle of which is based on quasielastic light scattering (QELS).

This index may vary from 0.01 to 0.35 and in particular can vary from 0.05 to 0.35.

For the capsules with a mean size of greater than one micrometre, the particle size distribution may be characterized by a mean size and a uniformity coefficient measured using a laser diffraction particle sizer, such as, for example, the Master Sizer 2000® from Malvern.

The capsules in accordance with the invention, the size of which is greater than a micrometre, may have a uniformity coefficient advantageously of less than or equal to 0.45 and preferably of greater than or equal to 0.1.

According to one embodiment, a process of the invention may result in a suspension of capsules of the invention being obtained which can exhibit a level of capsules varying from 3 to 90% by weight, in particular varying from 10 to 60% by weight, and more particularly varying from 20 to 50% by weight, with respect to the total weight of the suspension.

According to one embodiment, a suspension of capsules of the invention may comprise up to 80% by weight of encapsulated active agent with respect to the total weight of active agent employed in stage a) of the process described above, in particular up to 85%, in particular up to 90% and more particularly up to 95%, indeed even up to 99%, of encapsulated active agent, with respect to the total weight of active agent employed in the initial mixture of step a) of the above process.

The capsules obtained by a process of the invention advantageously have an improved leaktightness.

The leaktightness of the capsules of the invention may be measured by any technique known to a person skilled in the art in the field, such as the measurement of the leakage from the capsules of a coloured or fluorescent marker encapsulated beforehand.

Process

As indicated above, a subject-matter of the present invention is a process for the preparation of a suspension of capsules of core/shell type comprising at least the steps of:

a) having a heterogeneous mixture at ambient temperature (T_{amb}) comprising:
an oily phase,
identical or different polymers which are immiscible with water and with the oily phase, at least one polymer of which is an oligomer having a weight-average molecular weight of less than 10 000 and a hydroxyl number (N_{OH}) of greater than or equal to 10 mg KOH/g,

b) homogenizing the oily phase and the polymers by heating the mixture obtained in step a) at a homogenization temperature T_{H}, T_{H} being less than 100° C., the said mixture having, at the temperature T_{H}, a viscosity of less than 17 mPa·s,

c) dispersing, at the temperature T_{H}, the homogeneous mixture obtained in step b) in an aqueous phase in order to obtain a direct preemulsion, the oily phase of which comprises the polymers,

d) subjecting the preemulsion obtained in step c) to a combination of shear forces appropriate for the reduction in the diameter of the particles of the dispersed mixture to a mean size of less than or equal to approximately 50 μm,

e) cooling the emulsion obtained in step d) to a temperature T_{c} suitable for the coacervation of the said polymers and for the coating of drops of the said oily phase by the coacervates, and

f) cooling the suspension obtained in step e) to a temperature Tₚ suitable for the formation of the expected capsules by the precipitation and/or crystallization of the coacervates,

the oily phase being suitable for the formation of a homogeneous mixture with the said polymers, at a temperature T_{H}, and for the appearance of a phenomenon of coacervation of the said polymers at a temperature T_{c}.

According to a specific embodiment, the polymers employed in step a) are oligomers having a weight-average molecular weight of less than 10 000 and a hydroxyl number (N_{OH}) of greater than or equal to 10 mg KOH/g.

The mixture obtained in step a) is produced at ambient temperature, varying from 20 to 25° C., making it possible to observe the heterogeneous nature of the mixture.

In order to obtain such a mixture, the polymers and the oily phase are selected so that:
an oily phase and polymers mixture has a homogenization temperature T_{H} of less than 100° C. and greater than ambient temperature T_{amb},
this mixture has a coacervation temperature Tc which is less than TH and greater than Tamb,
this mixture has, at the temperature TH, a viscosity of less than or equal to 17 mPa·s, and
the polymers, at ambient temperature, are immiscible with water and with the oily phase.

According to one embodiment, the oily phase may comprise at least one fat-soluble active agent and/or at least one fat-dispersible active agent.

According to one embodiment, the oily phase may be mixed beforehand with the active agent(s) to be encapsulated, before addition of the polymers.

According to one alternative embodiment, the oily phase, the active agent(s) and the polymers may be mixed simultaneously.

The fat-soluble active agents capable of being employed in the invention are chosen and employed in contents such that the properties of the tripartite mixture thus obtained (oily phase/polymers/fat-soluble active agents) are in accordance with the required properties described above for the bipartite mixture obtained in stage a) (oily phase/polymers).

Within the meaning of the invention, the term “heterogeneous mixture” is understood to mean a mixture comprising at least two different phases which are visible to the naked eye.

The heterogeneous mixture thus obtained in step a) is subsequently heated to a homogenization temperature T_{H} of the mixture. Such a temperature can be easily determined by routine experiments known to a person skilled in the art.

The heating means suitable for the implementation of the invention are commonly used by a person skilled in the art in the field.

Examples of polymers, in particular of oligomers, of oily phase and, if appropriate, of active agent(s) suitable for the invention are in particular presented subsequently.

The homogenization temperature of a mixture of the invention is greater than or equal to the temperature at which the polymers, in particular the oligomers, become miscible with the oily phase, and less than 100° C.

Thus, on conclusion of step b), a single-phase homogeneous mixture is obtained.

From the viewpoint of the invention, the homogeneous nature of such a mixture is assessed with regard to the oily phase and the polymers, in particular the oligomers, and, if appropriate, the fat-soluble active agents.

According to an alternative embodiment, the homogeneity of a mixture of the invention comprising at least one fat-dispersible active agent is assessed, on the one hand, by the homogeneity of the oily phase/oligomers mixture and, on the other hand, the homogeneity of the dispersion of the fat-dispersible active agents.

The miscibility of the polymers, in particular of the oligomers, of the invention in an oily phase appropriate for the process of the invention, optionally comprising one or more fat-soluble active agent(s), and also, if appropriate, the homogeneous dispersion of the fat-dispersible active agents can, for example, be assessed with the naked eye.

The mixture obtained in step b) has, at the temperature T_{H}, a viscosity of less than or equal to 17 mPa·s.

The viscosity of a mixture of the invention can be measured using an RS150, Rhéostress rheometer from Kaake equipped with cone/plate geometry (diameter of 60 mm, angle of 2°, made of titanium) under flow (shear gradient scanning from 1 to 1000 s⁻¹).

The viscosity value recorded is that on the newtonian plateau with a low shear gradient.

For example, a mixture obtained in step b) may have, at the temperature T_{H}, a viscosity varying from 1 to 17 mPa·s.

In step c), the single-phase mixture is subsequently dispersed in an aqueous phase heated to a temperature such that the temperature of the mixture thus obtained is greater than or equal to the temperature T_{H}, so as to obtain a preemulsion of drops of the mixture in a continuous aqueous phase.

According to one embodiment, the aqueous phase may be heated to a temperature equivalent to the temperature T_{H} determined above.

According to one embodiment, as indicated below, one or more surface-active agents as defined below may be introduced into the oily phase and/or into the aqueous phase, before the preparation of the preemulsion.

If appropriate, the additional surface-active agent(s) may be employed in order to obtain a lamellar phase intended to surround the capsules of the invention.

The devices which make it possible to prepare a preemulsion according to the invention are those commonly employed by a person skilled in the art in the field.

The expected final size of the capsules is generally determining for the choice of the device and forces to be employed during the preparation of the preemulsion. Thus, the device and the conditions for the preparation of the preemulsion of step c) are adjusted by a person skilled in the art from the viewpoint of the size of the capsules of the invention to be obtained.

The preemulsion obtained in step c) may be subjected, in step d), to shear forces sufficient to produce drops of the dispersed mixture at most equal to 50 μm in diameter, in particular at most equal to 10 μm in diameter.

Step d) may be carried out by any device known to a person skilled in the art suitable for the reduction in the size of the globules of the preemulsion to the size expected for the capsules of the invention, in particular less than or equal to 50 μm.

Mention may be made, as example of device suitable for the invention, of high-pressure homogenizers or ultrasonic homogenizers.

The emulsion obtained in stage d) is then at least cooled to a coacervation temperature T_{c}, with gentle stirring (stage e)).

The coacervation temperature T_{c} is the temperature at which the polymers, and in particular the oligomers, organize themselves into coacervates, that is to say in the form of soft shells, around droplets of the oily phase. T_{c} is greater than T_{amb}.

Advantageously, T_{c} may be greater than or equal to 45° C.

The suspension of oily particles coated with a coacervate of oligomers is subsequently subjected to a step of cooling f) down to a temperature Tₚ suitable for the formation of the expected capsules by the precipitation and/or crystallization of the coacervates.

The temperature Tₚ is less than the lowest melting point Mp of the oligomers employed in a process of the invention.

According to one embodiment, the polymers, and in particular the oligomers, are advantageously chosen so that Mp is greater than T_{mab}. Advantageously, when Mp is greater than T_{amb}, Tₚ can be chosen equal to T_{amb}.

According to another embodiment, the polymers, and in particular the oligomers, may be chosen so that Mp is less than T_{amb}. Advantageously, when Mp is less than T_{amb}, it is possible to carry out an additional stage g) of crosslinking, as described below.

Steps e) and f) of cooling an emulsion of the invention may be carried out continuously or stepwise.

The term “continuous cooling” is understood to mean the maintenance of the emulsion to be cooled for an identical period of time at each specific and identical lower temperature fraction.

The term “stepwise cooling” is understood to mean the maintenance of the emulsion to be cooled at a lower temperature fraction for a given period of time.

According to one embodiment, a mixture according to the invention comprising an oily phase, if appropriate comprising the active agent(s), and polymers, in particular oligomers, is formulated so that the temperature T_{c} is greater than or equal to 45° C.

According to an alternative embodiment, as many steps as desired may be introduced into the cooling process, before or after T_{c} and Tₚ.

According to an alternative embodiment, the cooling rate may be constant, accelerated or slowing down from T_{H} to T_{c} and/or from T_{c} to Tₚ.

According to another embodiment, the cooling of the emulsion of the invention may be carried out continuously from T_{H} to Tₚ.

The cooling may be carried out by confining the emulsion thus obtained to a chamber, the temperature of which can be set so as to regulate the cooling rate and the various cooling stages.

According to an alternative embodiment, the cooling may be carried out by placing the emulsion obtained in step d) in an atmosphere maintained at ambient temperature.

On conclusion of the cooling step, a suspension of capsules is then obtained, the size of which can be monitored using a light scattering laser particle sizer or a laser diffraction particle sizer, as indicated above.

According to one embodiment, a process according to the invention can additionally comprise a step g) of crosslinking oligomers.

The optional crosslinking step can make it possible to reinforce the wall of the capsules of the invention. Such a step advantageously makes it possible to improve the leaktightness of the capsules of the invention.

The crosslinking step may be carried out by means of crosslinking agent(s) as defined below.

According to one embodiment, the crosslinking agent(s) is/are added to the emulsion of the invention on conclusion of the cooling step.

According to one embodiment, the crosslinking step g) may in particular be carried out when the polymer(s), in particular the oligomer(s), used in the process of the invention has/have a melting point Mp which is less than or equal to 45° C., in particular less than or equal to T_{amb}, and especially is/are liquid at ambient temperature.

According to one embodiment, the polymers, in particular the oligomers, and the oily phase, optionally comprising one or more active agent(s), may be present according to a ratio by weight varying from 5/1 to 1/20, in particular varying from 2/1 to 1/20 and more particularly varying from 2/1 to 1/10.

According to one embodiment, the oily phase and the active agent(s) may be present in a heterogeneous mixture according to the invention according to a ratio by weight varying from 100/0 to 1/99.

According to one embodiment, the oily phase can form the active agent.

Polymers

Within the meaning of the invention, the term “polymers” is understood to mean compounds comprising at least 2, in particular at least 3 and more particularly at least 4 identical repeat units.

Within the meaning of the invention, the term “oligomer” is understood to mean polymers comprising at most 100 monomer units.

The oligomers suitable for the invention can have a weight-average molecular weight of less than 10 000 and greater than or equal to 400.

In particular, they can have a hydroxyl number (N_{OH}) at least equal to 10 mg KOH/g, in particular varying from 10 to 700 mg KOH/g, in particular varying from 10 to 600 mg KOH/g and more particularly varying from 40 to 500 mg KOH/g.

In one implementation of a process of the invention, the oligomers can be identical or different.

The polymers suitable for the invention, in particular the oligomers, are immiscible with water and with the oily phase intended to form the core of the capsules of the invention. They have a melting point Mp of less than 100° C., in particular varying from −20° C. to 95° C. and more particularly varying from −10° C. to 80° C.

According to one embodiment, it is possible to employ, in step a), as a mixture with oligomers having a weight-average molecular weight of less than 10 000, polymers having a weight-average molecular weight of greater than or equal to 10 000, in particular of greater than or equal to 15 000.

In particular, the polymers with a molecular weight of greater than or equal to 10 000 have a molecular weight not exceeding 80 000.

According to an alternative embodiment, the polymers employed in a process of the invention all have a weight-average molecular weight of less than 10 000.

Mention may be made, by way of illustration and without implied limitation of oligomers suitable for the invention, for example, of the oligomers selected from the group consisting in polycaprolactones, poly-L-lactides, poly-DL-lactides, polyglycolides and their copolymers, polymers of 3-hydroxybutyric acid and its copolymers with hydroxyvaleric acid, polycarbonate diols, polyalkylene adipates, polyester polyols, dendritic polyesters comprising an end hydroxyl functional group, and their blends.

The oligomers suitable for the invention can, for example, be selected from the group consisting in:
polycaprolactones, for example those sold by Solvay, which can be obtained from a diol, a triol, a tetraol, or a carboxylated glycol.

The diols used for the preparation of these polycaprolactone oligomers are: 1,4-butanediol, 1,6-hexanediol, mono- or diethylene glycol, 2,2-dimethyl-1,3-propanediol (neopentyl glycol), or dimethylolpropionic acid. The triols can, for example, be a mixture of diethylene glycol and of glycerol, trimethylolpropane.
poly-L-lactides, poly-DL-lactides, polyglycolides and their copolymers,
polymers of 3-hydroxybutyric acid and its copolymers with hydroxyvaleric acid,
polycarbonate diols, such as hexanediol polycarbonates, for example UH-Carb 50 and 300 sold by UBE,
polyalkylene adipates comprising linear or branched homopolymers of adipic acid and of an alkanediol and copolymers of poly(ester ether) type, obtained from adipic acid and from one or more alkanediols and/or ether diols and/or triols; the alkanediols used for the preparation of the said polyalkylene adipates can be linear- or branched-chain C₂-C₆ alkanediols selected from the group consisting in ethylene glycol, propylene glycol, 1,3-propanediol, 1,4-butanediol, 1,5-pentanediol, 1,6-hexanediol and neopentyl glycol. The ether diols are di-, tri- or tetra(C₂-C₄ alkylene) glycols, such as diethylene glycol, triethylene glycol, tetraethylene glycol, dipropylene glycol, tripropylene glycol, tetrapropylene glycol, dibutylene glycol, tributylene glycol or tetrabutylene glycol; mention may be made, by way of example, of the Fomrez® products sold by Witco and the polyethylene adipates from Scientific Polymer Products; mention may also be made, by way of example, of Eastar Bio® from Eastman Chemical (poly(tetramethylene adipate-co-terephthalate)) and Ecoflex F BX 7011® from BASF (1,4-butanediol/terephthalic acid/adipic acid terpolymer).

Mention may in particular be made, as example of polyalkylene adipates suitable for the invention, of the Fomrez® products from Witco.
polyester polyols, such as the copolymers obtained by polycondensation of at least one aliphatic dicarboxylic acid with at least two alkanediols, or with at least one alkanediol and at least one (hydroxyalkyl)alkanediol, and optionally a small proportion of triols.

The aliphatic dicarboxylic acid used for the preparation of the said polyester polyols may be selected, for example, from the group consisting in malonic acid, succinic acid, glutaric acid, adipic acid (or hexane-1,6-dioic acid), pimelic acid, sebacic acid, azelaic acid and their mixtures. According to one embodiment of the invention, the dicarboxylic acid can be adipic acid.

The alkanediols used for the preparation of the polyester polyols may be selected from the group consisting in linear- or branched-chain alkanediols comprising from 2 to 20 carbon atoms and preferably from 2 to 10 carbon atoms. They may be selected in particular from the group consisting in ethylene glycol, propylene glycol, 1,3-propanediol, 1,4-butanediol, 1,5-pentanediol, 1,6-hexanediol, neopentyl glycol and their mixtures. According to one embodiment of the invention, the alkanediol may be selected chosen from the group consisting in 1,4-butanediol, 1,6-hexanediol and their mixtures. For example, the alkanediol can be 1,4-butanediol.

In the present patent application, the term “(hydroxyalkyl)alkanediols optionally comprising an alkyl chain” is understood to mean alkanediols comprising at least one hydroxyalkyl group which can in addition comprise an alkyl chain, where the hydroxyalkyl group and the alkyl chain are, independently of one another, saturated linear or branched chains comprising from 1 to 10 carbon atoms. The (hydroxyalkyl)alkanediols which can be used to form the polyester polyols of the present invention can be selected, for example, from the group consisting in 2-alkyl-2-(hydroxyalkyl)-1,3-propanediol compounds, where the hydroxyalkyl group and the alkyl chain comprise, independently of one another, from 1 to 10 carbon atoms, such as, for example, 2-ethyl-2-(hydroxymethyl)-1,3-propanediol and 2-methyl-2-(hydroxymethyl)-1,3-propanediol; 2-(hydroxyalkyl)-1,3-propanediol compounds where the hydroxyalkyl group comprises from 1 to 10 carbon atoms; and their mixtures.

According to one embodiment of the invention, use may be made, as (hydroxyalkyl)alkanediol, of 2-ethyl-2-(hydroxymethyl)-1,3-propanediol.

The polyester polyols suitable for the invention can also comprise a limited number of branching units derived from triols. The triols used can be chosen from glycerol, trimethylolethane and trimethylolpropane. The fraction of the branching units derived from the above triols generally does not exceed 5 mol %, with respect to the combined units derived from diols and from triols.

According to one embodiment of the present invention, the polyester polyols may be selected from the group consisting in the polyester polyols obtained from adipic acid, 1,4-butanediol and 1,6-hexanediol and the polyester polyols obtained from adipic acid, 1,4-butanediol and 2-ethyl-2-(hydroxymethyl)-1,3-propanediol. Mention may be made, as polyester polyols suitable for the invention, for example, of those sold by Inolex under the Lexorez names. Particular preference is given to those obtained from adipic acid, 1,4-butanediol and 2-ethyl-2-(hydroxymethyl)-1,3-propanediol.

The polyester polyols suitable for the invention may have a melting point ranging from −20° C. to 95° C. and in particular from −10° C. to 80° C.

The polyester polyols suitable for the invention may be prepared according to processes commonly used for the preparation of polyesters.

The polyester polyols suitable for the invention can in particular be those sold under the Lexorez® name by Inolex and the polyester polyols sold under the Eternacoll name, such as Eternacoll 3020, from UBE.
polylactate/ricinoleate copolymers, such as those sold under the trade name Ethox PLPR by Ethox Chemical, and polylactate/hydroxystearate copolymers, in particular those sold under the trade mark Ethox PLPHS by Ethox Chemical,
dendritic polyesters comprising an end hydroxyl functional group.

Dendritic polymers or dendrimers (from the Greek dendron=tree) are “arborescent”, that is to say highly branched, polymeric molecules invented by D. A. Tomalia and his team at the beginning of the 1990s (Donald A. Tomalia et al., Angewandte Chemie, Int. Engl. Ed., vol. 29, No. 2, pages 138-175). They are molecular structures constructed around a central unit which is generally polyvalent. Branched chain-extending units are connected, around this central unit, in concentric layers and according to a perfectly predetermined structure, thus giving rise to symmetrical monodisperse macromolecules having a well defined chemical and stereochemical structure.

The dendritic polymers suitable for the invention are hyperbranched polymers which have the chemical structure of a polyester and which are terminated by hydroxyl groups optionally modified by at least one chain-terminating agent. The structure and the preparation of such polymers are described in Patent Applications WO-A-93/17060 (or its equivalent U.S. Pat. No. 5,418,301) and WO 96/12754 (or its equivalent U.S. Pat. No. 5,663,247), the content of which are herein incorporated by reference.

The dendritic polymers used in the present invention can be defined as being highly branched macromolecules of polyester type composed:
of a central unit derived from an initiator compound carrying one or more hydroxyl functional groups (a),
of chain-extending units derived from a chain-extending molecule carrying a carboxyl functional group (b) and at least two hydroxyl functional groups (c),
   each of the hydroxyl functional groups (a) of the central molecule being the starting point for a polycondensation (esterification) reaction which begins with the reaction of the hydroxyl functional groups (a) of the central molecule with the carboxyl functional groups (b) of the chain-extending molecules and then continues by reaction of the carboxyl functional groups (b) with the hydroxyl functional groups (c) of the chain-extending molecules.

A “generation X” dendrimer is the name for a hyperbranched polymer prepared by X condensation cycles, each cycle consisting in reacting all the reactive functional groups of the central unit or of the polymer with one equivalent of a chain-extending molecule.

The initiator compound carrying one or more hydroxyl functional groups which forms the central unit around which the dendritic structure will be constructed is a mono-, di- or polyhydroxylated compound. It may be selected from the group consisting in:

(a) a monofunctional alcohol,

(b) an aliphatic, cycloaliphatic or aromatic diol,

(c) a triol,

(d) a tetraol,

(e) a sugar alcohol,

(f) anhydro-ennea-heptitol or dipentaerythritol,

(g) an α-alkylglycoside,

(h) a polyalkoxylated polymer obtained by polyalkoxylation of one of the alcohols (a) to (g), having a molar mass at most equal to 8000.

Mention may be made, as examples of initiator compounds suitable for the preparation of the dendritic polyesters suitable for the invention, of ditrimethylolpropane, ditrimethylolethane, dipentaerythritol, pentaerythritol, an alkoxylated pentaerythritol, trimethylolethane, trimethylolpropane, an alkoxylated trimethylolpropane, glycerol, neopentyl glycol, dimethylolpropane or 1,3-dioxane-5,5-dimethanol.

These hydroxylated initiator compounds, forming the central unit of the future dendrimer, are reacted with molecules, referred to as chain-extending molecules, which are compounds of diol-monoacid type chosen from:
monocarboxylic acids comprising at least two hydroxyl functional groups, and
monocarboxylic acids comprising at least two hydroxyl functional groups, one or more of which carry/ies a hydroxyalkyl substituent.

Examples of such compounds are dimethylolpropionic acid, α,α-bis(hydroxymethyl)butyric acid, α,α,α-tris(hydroxymethyl)acetic acid, α,α-bis(hydroxymethyl)valeric acid, α,α-dihydroxypropionic acid and 3,5-dihydroxybenzoic acid.

According to one embodiment of the invention, the initiator compound can be selected from the group consisting in trimethylolpropane, pentaerythritol and an ethoxylated pentaerythritol and the chain-extending molecule is dimethylolpropionic acid.

A portion of the end hydroxyl functional groups of the dendritic polymers of polyester type can carry substituents derived from at least one chain-terminating agent.

The fraction of these end hydroxyl functional groups carrying a chain-terminating unit is generally between 1 and 90 mol %, in particular between 10 and 50 mol %, with respect to the total number of end hydroxyl functional groups.

The choice of an appropriate chain-terminating agent makes it possible to modify to perfection the physicochemical properties of the dendritic polyesters used in the invention.

The said chain-terminating agent may be chosen from a great variety of compounds capable of forming covalent bonds with the end hydroxyl functional groups.

These compounds can comprise:

i) the saturated or unsaturated aliphatic or cycloaliphatic monocarboxylic acids (or anhydrides),

ii) saturated or unsaturated fatty acids,

iii) aromatic monocarboxylic acids,

iv) monomeric or oligomeric diisocyanates or their addition products,

v) epihalohydrins,

vi) glycidyl esters of a monocarboxylic acid or of a C₁₋₂₄ fatty acid,

vii) glycidyl ethers of C₁₋₂₄ monovalent alcohols,

viii) addition products derived from a saturated or unsaturated aliphatic or cycloaliphatic mono-, di- or polycarboxylic acid or from the corresponding anhydrides,

ix) addition products derived from an aromatic mono-, di- or polycarboxylic acid or from the corresponding anhydrides,

x) epoxides of an unsaturated C₃₋₂₄ monocarboxylic acid or of a corresponding triglyceride,

xi) saturated or unsaturated monofunctional aliphatic or cycloaliphatic alcohols,

xii) monofunctional aromatic alcohols,

xiii) addition products derived from a saturated or unsaturated mono-, di- or polyfunctional aliphatic or cycloaliphatic alcohol, and

xiv) addition products derived from a mono-, di- or polyfunctional aromatic alcohol.

Mention may be made, as example of chain-terminating agents, of lauric acid, linseed oil fatty acids, soybean oil fatty acids, tallow fatty acids, dehydrogenated castor oil fatty acids, crotonic acid, capric acid, caprylic acid, acrylic acid, methacrylic acid, benzoic acid, para-(tert-butyl)benzoic acid, abietic acid, sorbinic acid, 1-chloro-2,3-epoxypropane, 1,4-dichloro-2,3-epoxybutane, epoxidized soybean oil fatty acids, trimethylolpropane diallyl ether maleate, 5-methyl-1,3-dioxane-5-methanol, 5-ethyl-1,3-dioxane-5-methanol, trimethylolpropane diallyl ether, pentaerythritol triallyl ether, pentaerythritol triacrylate, pentaerythritol triethoxylate triacrylate, toluene 2,4-diisocyanate, toluene 2,6-diisocyanate, hexamethylene diisocyanate or isophorone diisocyanate.

Mention may be in particular be made, among these chain-terminating agents, of capric acid and caprylic acid or a mixture of these.

The dendritic polymers of polyester type comprising end hydroxyl functional groups and optionally carrying chain-terminating groups are known and are sold by Perstorp.

Polymers suitable for the present invention can be:
a dendritic polyester obtained by polycondensation of dimethylolpropionic acid with trimethylolpropane which is devoid of chain-terminating agents, for example that sold under the name “Boltorn® H40 (TMP core)” by Perstorp;
a dendritic polyester obtained by polycondensation of dimethylolpropionic acid with polyoxyethylenated pentaerythritol (on average 5 ethylene oxide units on each hydroxyl functional group) which is devoid of chain-terminating agent, for example that sold under the name “Boltorn® 130” by Perstorp;
a generation 3 dendritic polyester obtained by polycondensation of dimethylolpropionic acid with polyoxyethylenated pentaerythritol (on average 5 ethylene oxide units on each hydroxyl functional group), 50% of the hydroxyl functional groups of which are esterified with C₈₋₁₀ acids and in particular capric (C₁₀) acid and caprylic (C₈) acid (“Boltorn® H30 (esterified)” sold by Perstorp).

Mention may be made, as dendritic polyesters comprising an end hydroxyl functional group suitable in particular for the invention, of the oligomers sold under the Boltorn® name from Perstorp).

Mention may in particular be made, among oligomers suitable for the invention, of polycaprolactones, such as Capa® HC 1100, with a molecular weight of 1000, a hydroxyl number (mg KOH/g) of 110 and a melting point (° C.) of 45-50, Capa® 2200A and Capa® 2201, with a molecular weight of 2000, a hydroxyl number (mg KOH/g) of 56 and a melting point (° C.) of 40-50, Capa® 2302A and Capa® 2303, with a molecular weight of 3000, a hydroxyl number (mg KOH/g) of 37 and a melting point (° C.) of 50-60, or Capa® 3050, with a molecular weight of 540, a hydroxyl number (mg KOH/g) of 310 and a melting point (° C.) of 0-0; polyalkylene adipates, such as Fomrez® F930, with a molecular weight of 2000, a hydroxyl number (mg KOH/g) of 54-58 and a melting point (° C.) of 50; polyester polyols, such as Lexorez® 1151-35, with a molecular weight of 3200, a hydroxyl number (mg KOH/g) of 35 and a melting point (° C.) of 55-65, Lexorez® 1460-36, with a molecular weight of 3200, a hydroxyl number (mg KOH/g) of 36 and a melting point (° C.) of 40-50, UH-Carb® 300, with a molecular weight of 3000, a hydroxyl number (mg KOH/g) of 37 and a melting point (° C.) of 52, UH-Carb 50, with a molecular weight of 500, a hydroxyl number (mg KOH/g) of 224 and a melting point (° C.) of 33, and Eternacoll® 3020, with a molecular weight of 3500, a hydroxyl number (mg KOH/g) of 29-35 and a melting point (° C.) of 65.

Mention may be made, as examples of polymers having a weight-average molecular weight of greater than or equal to 10 000 and in particular of greater than or equal to 15 000, of the polymers selected from the group consisting in:
C₂-C₁₂ and in particular C₂-C₆ alkyl cyanoacrylate polymers with the alkyl radical being able in particular to be chosen from the ethyl, n-butyl, hexyl, isobutyl and isohexyl radicals,
polymers formed by poly-L-lactides, poly-DL-lactides, polyglycolides and the corresponding copolymers, such as copoly(DL-lactides and glycolides), copoly(glycolides and caprolactones) and the like,
polycaprolactones, such as polycaprolactones having a melting point varying from 40 to 70° C. and with a molecular weight of between 2000 and 100 000, such as, for example, those sold by Solvay under the commercial references Capa 6806 (MW of 80 000), Capa 6100 (MW of 10 000), Capa 6506 (MW of 50 000), Capa 6250 (MW of 25 000), Capa 2803 (MW of 8000) or Capa 2403 D (MW of 4000),
polymers of 3-hydroxybutyric acid and its copolymers with hydroxyvaleric acid,
copolymers of vinyl chloride and of vinyl acetate, for example those sold under the name Rhodopas AX 8515 by Rhône-Poulenc,
copolymers of methacrylic acid and ester, in particular of methacrylic acid and of methacrylic acid ester, for example those sold under the name Eudragit L 100® by Röhm Pharma,
polyvinyl acetate phthatate,
cellulose acetate phthalate,
polyvinylpyrrolidone/vinyl acetate copolymer,
polyethylene/vinyl acetates,
polyacrylonitriles,
polyacrylamides,
polyethylene glycols,
poly(C₁ to C₄ hydroxyalkyl methacrylate)s and in particular poly(hydroxyethyl methacrylate)s,
cellulose derivatives, such as esters of cellulose and of at least one C₁ to C₄ carboxylic acid, in particular mixed cellulose esters of two types of carboxylic acids,
polystyrene and copolymers of styrene and of maleic anhydride, copolymers of styrene and of acrylic acid, styrene-ethylene/butylene-styrene block terpolymers or styrene-ethylene/propylene-styrene block terpolymers,
styrene alkyl alcohol oligomers,
terpolymers of ethylene, of vinyl acetate and of maleic anhydride,
polyamides,
polyethylenes,
polypropylenes,
organopolysiloxanes, including polydimethylsiloxanes,
polyalkylene adipates, which encompass both linear or branched homopolymers of adipic acid and of an alkanediol and copolymers of poly(ester ether) type, obtained from adipic acid and from one or more alkanediols and/or ether diols and/or triols; the alkanediols used for the preparation of the said polyalkylene adipates can be linear- or branched-chain C₂-C₆ alkanediols chosen from ethylene glycol, propylene glycol, 1,3-propanediol, 1,4-butanediol, 1,5-pentanediol, 1,6-hexanediol and neopentyl glycol. The ether diols are di-, tri- or tetra(C₂-C₄ alkylene) glycols, such as diethylene glycol, triethylene glycol, tetraethylene glycol, dipropylene glycol, tripropylene glycol, tetrapropylene glycol, dibutylene glycol, tributylene glycol or tetrabutylene glycol; mention may more particularly be made, by way of example, of the Fomrez® products sold by Witco and the polyethylene adipates from Scientific Polymer Products,
polyester polyols obtained by polycondensation of an aliphatic dicarboxylic acid with at least two alkanediols or with at least one alkanediol and at least one (hydroxyalkyl)alkanediol, with the aliphatic dicarboxylic acid being able to be adipic acid (hexane-1,6-dioic acid); such polymers are described in particular in the document FR 2 836 381 (or US 2003-224060), the content of which being incorporated herein by reference.
polysilsesquioxane silicone polymers, in particular a polyalkylsilsesquioxane of formula (R—SiO_{3/2})ₓ₀, in which R represents a saturated or unsaturated and linear, branched or cyclic hydrocarbon radical; for example of the —CₙH₂ₙ₊₁ type, with n being an integer ranging from 1 to 20, in particular a methyl, ethyl, propyl, butyl, pentyl, hexyl, heptyl, octyl, nonyl, decyl, dodecyl, tridecyl, tetradecyl, hexadecyl, octadecyl and eicosyl radical; or also an aryl group, in particular phenyl or tolyl; a cycloalkyl group, in particular cyclobutyl, cyclopentyl or cyclohexyl; an alkenyl group, in particular vinyl or allyl; an aralkyl group, in particular 2-phenylethyl or benzyl; R also being able to comprise one or more halogen atoms, in particular fluorine or chlorine atoms; preferably, R is a methyl, ethyl, propyl or phenyl radical; and x is a number of units and can be between 1 and 10, in particular 1 to 4; mention may be made, as example, of Belsil PMS MK® from Wacker or Resin KR 220® from Shin-Etsu,
dendritic polyesters comprising an end hydroxyl functional group, in particular those described in document FR 2 790 405 (or U.S. Pat. No. 6,379,683), the content of which being incorporated herein by reference.
polymers which are dispersible in water but nevertheless soluble in water-immiscible solvents, such as, for example: polyesters, poly(ester amide)s, polyurethanes and vinyl copolymers carrying carboxylic and/or sulphonic acid functional groups, in particular those described in the document FR 2 787 729, the content of which being incorporated herein by reference.
block copolymers which are insoluble in water at ambient temperature and solid at ambient temperature, having at least one block of one of the above polymers, and
their blends.

When they are present, the polymers having a weight-average molecular weight of greater than or equal to 10 000 are employed in a content at most equal to 20% by weight, with respect to the total weight of the composition, in particular from 1 to 15% by weight and more particularly from 1 to 10% by weight, with respect to the total weight of the composition.

Oily Phase

The term “oily phase” is understood to denote, within the meaning of the present invention, a phase comprising at least one oil.

An oily phase according to the invention can comprise a fat-soluble or fat-dispersible active agent or can itself form an active agent. Thus, if appropriate, an oily phase suitable for the invention makes it possible to dissolve fat-soluble active agents and/or to disperse fat-dispersible active agents.

According to one embodiment, an oily phase suitable for the invention may comprise an oil having a high polarity, such as, for example, isopropyl N-lauroylsarcosinate (Eldew SL 205) from Ajinomoto, in order to confer, on the mixture of the invention, a homogeneity temperature T_{H} as defined above.

According to another embodiment, an oily phase suitable for the invention may comprise an oil having a low polarity, such as, for example, an alkane, for example hydrogenated polyisobutene (sold under the Parléarm® trade name by NOF) or hydrogenated 6-8 mol isoparaffin, in order to confer, on the mixture of the invention, a coacervation temperature T_{c} as defined above.

An oily phase suitable for the invention may comprise at least one oil chosen from nonvolatile oils, and their mixtures.

The oils suitable for the implementation of the invention can be chosen from vegetable oils, animal oils, mineral oils or synthetic oils, and their mixtures.

They can be of hydrocarbon type, such as, for example, triglycerides, esters, alkanes or polyolefins, of silicone type or of fluorinated type and may or may not be modified.

The term “hydrocarbon oil” is understood to denote an oil comprising mainly hydrogen and carbon atoms and optionally oxygen, nitrogen, sulphur and/or phosphorus atoms.

Within the meaning of the present invention, the term “fluorinated oil” is understood to mean an oil comprising at least one fluorine atom.

Within the meaning of the present invention, the term “silicone oil” is understood to mean an oil comprising at least one silicon atom and in particular at least one Si—O group.

According to one embodiment, they can be used alone or as a mixture, with one another or with other compounds as defined, for example, subsequently.

The nonvolatile oils can be chosen in particular from nonvolatile hydrocarbon oils, if appropriate fluorinated, and/or nonvolatile silicone oils.

Mention may in particular be made, as nonvolatile hydrocarbon oil, of:
hydrocarbon oils of animal origin, such as squalane;
hydrocarbon oils of vegetable origin, such as phytosteryl esters, for example phytosteryl oleate, phytosteryl isostearate and lauroyl/octyldodecyl/phytosteryl glutamate (Ajinomoto, Eldew PS203); triglycerides composed of esters of fatty acids and of glycerol, the fatty acids of which can have varied chain lengths from C₄ to C₂₄, it being possible for these chains to be linear or branched and saturated or unsaturated; these oils are in particular heptanoic or octanoic triglycerides; wheat germ, sunflower, grape seed, sesame, maize, apricot, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; shea butter; or triglycerides of caprylic/capric acids, such as those sold by Stéarineries Dubois or those sold under the names Miglyol 810®, 812® and 818® by Dynamit Nobel, and their mixtures;
linear or branched hydrocarbons of mineral or synthetic origin, such as liquid petrolatum, polydecenes, hydrogenated polyisobutene, such as parleam, and their mixtures;
synthetic ethers having from 10 to 40 carbon atoms;
synthetic esters, such as oils of formula R₁COOR₂ in which R₁ represents the residue of a linear or branched fatty acid comprising from 1 to 40 carbon atoms, and R₂ represents a hydrocarbon chain, in particular a branched hydrocarbon chain, comprising from 1 to 40 carbon atoms, provided that R₁+R₂≧10;
and their mixtures.

The esters can in particular be chosen from fatty acid esters, such as, for example:
cetearyl octanoate, esters of isopropyl alcohol, such as isopropyl myristate, isopropyl palmitate or isopropyl lauroylsarcosinate (Eldew SL 205, from Ajinomoto), ethyl palmitate, 2-ethylhexyl palmitate, isopropyl stearate or isostearate, isostearyl isostearate, isocetyl stearate, octyl stearate, hydroxylated esters, such as isostearyl lactate or octyl hydroxystearate, diisopropyl adipate, heptanoates and in particular isostearyl heptanoate, octanoates, decanoates or ricinoleates of alcohols or of polyalcohols, such as propylene glycol dioctanoate, cetyl octanoate, tridecyl octanoate, 2-ethylhexyl 4-diheptanoate, polyethylene glycol diheptanoate, propylene glycol di(2-ethylhexanoate) and their mixtures, hexyl laurate, esters of neopentanoic acid, such as isodecyl neopentanoate, isotridecyl neopentanoate, isostearyl neopentanoate or octyldodecyl neopentanoate, esters of isononanoic acid, such as isononyl isononanoate, isotridecyl isononanoate or octyl isononanoate, hydroxylated esters, such as isostearyl lactate or diisostearyl malate, alkyl benzoate, C₁₂ to C₁₅ alkyl benzoates, and their mixtures;
esters of polyols and esters of pentaerythritol, such as dipentaerythritol tetrahydroxystearate/tetraisostearate;
esters of dimer diols and dimer diacids, such as Lusplan DD-DA5® and Lusplan DD-DA7®, and their mixtures, sold by Nippon Fine Chemical and described in Application FR 2 851 915 (or US 2004-175338) filed on 6 Mar. 2003, the content of which is incorporated in the present patent application by way of reference;
fatty alcohols which are liquid at ambient temperature with a branched and/or unsaturated carbon chain having from 12 to 26 carbon atoms, such as 2-octyldodecanol, isostearyl alcohol, oleyl alcohol, 2-hexyldecanol, 2-butyloctanol and 2-undecylpentadecanol;
liquid higher fatty acids, such as oleic acid, linoleic acid, linolenic acid and their mixtures;
dialkyl carbonates, it being possible for the 2 alkyl chains to be identical or different, such as dicaprylyl carbonate, sold under the name Cetiol CC® by Cognis; and
their mixtures.

The nonvolatile silicone oils which can be used in the composition according to the invention can be nonvolatile polydimethylsiloxanes (PDMSs), such as simethicone, polydimethylsiloxanes comprising pendent alkyl or alkoxy groups and/or alkyl or alkoxy groups at the ends of the silicone chain, which groups each have from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyl-diphenyl)trisiloxanes and (2-phenylethyl)trimethylsiloxysilicates, dimethicones or phenyl trimethicones with a viscosity of less than or equal to 100 cSt, and their mixtures.

An oily phase suitable for the invention can optionally comprise at least one volatile oil, if appropriate chosen from volatile hydrocarbon oils, volatile silicones, volatile fluorinated oils and their mixtures.

According to one embodiment, an oily phase suitable for the invention can comprise at least one oil chosen from hydrocarbon oils of animal origin, hydrocarbon oils of vegetable origin, linear or branched hydrocarbons of mineral or synthetic origin, synthetic ethers having from 10 to 40 carbon atoms, synthetic esters of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂≧10, nonvolatile silicone oils, and their mixtures.

According to one alternative embodiment, an oily phase suitable for the invention can comprise at least one oil chosen from capric/caprylic acid triglycerides, isocetyl stearate, isopropyl N-lauroylsarcosinate and their mixtures.

According to one embodiment, an oily phase of isopropyl N-lauroylsarcosinate type can advantageously be employed with oligomers chosen from polycaprolactones, polyester polyol(s), polyalkylene adipates and mixtures of these.

Such a mixture can advantageously be employed for the encapsulation of Gatuline Derma Sensitive® (extract of caper buds in octyldodecyl myristate) from Gattefossé, shea liquid and/or solid fractions, Nutralipids® HY (mixture of passionflower, apricot kernel, maize and rice bran oil) from Nestle World Trade Corporation, and/or musk rose oils.

According to one embodiment, an oily phase of triglycerides of capric/caprylic acids type can advantageously be employed with oligomers chosen from polycaprolactones and mixtures of these.

Such a mixture can advantageously be employed for the encapsulation of active agents chosen from ginger extracts, vitamin E acetate and mixtures of these.

According to one embodiment, an oily phase of isocetyl stearate type can advantageously be employed with oligomers chosen from polyester polyol(s) and mixtures of these.

According to another embodiment, an oily phase comprising an oil mixture chosen from:
isopropyl N-lauroylsarcosinate and capric/caprylic acid triglycerides or
isopropyl N-lauroylsarcosinate and isocetyl stearate
   can advantageously be employed with oligomers chosen from polyester polyol(s), polyalkylene adipates and mixtures of these.

According to one embodiment, an oily phase suitable for the invention can comprise a fat-soluble active agent and/or a fat-dispersible active agent and/or be formed of an oily fat-soluble active agent as defined below.

Active Agents

An active agent suitable for the invention can be formed by the oily phase or can be dissolved or dispersed in an oily phase of the invention.

Mention may be made, as active agents suitable for the invention, of fat-soluble active agents selected from the group consisting in natural, vegetable, animal or synthetic oily substances which are liquid from 40° C., which have or do not have one or more known biological activities and which are insoluble in water (less than 2% by weight at ambient temperature), vegetable oils rich in unsaturations, such as borage oil or fish oils, sunscreens, vitamins E, F and K, their esters and their mixtures, vitamins such as vitamin A (retinol) or vitamin D, carotenes, such as β-carotene, salicylic acid, ginger extracts, musk rose oil, ceramides, α-linoleic acid, Gatuline Derma Sensitive® (extract of caper buds in octyldodecyl myristate), shea liquid and solid fractions, Nutralipids® HY (mixture of passionflower, apricot kernel, maize and rice bran oil), derivatives of these and mixtures of these. For example, use may be made, as salicylic acid derivatives, of those described in the documents FR-A-2 581 542 (or U.S. Pat. No. 4,767,750), EP-A-378 936 (U.S. Pat. No. 5,262,407) and EP-A-A-570 230 (or U.S. Pat. No. 5,580,549) the content of which being incorporated herein by reference, in particular 5-(n-octanoyl)salicylic acid, 5-(n-decanoyl)salicylic acid, 5-(n-dodecanoyl)salicylic acid, 5-(n-octyl)salicylic acid, 5-(n-heptyloxy)salicylic acid and 4-(n-heptyloxy)salicylic acid.

Mention may be made, as active agents suitable in particular for the invention, of Gatuline Derma Sensitive® (extract of caper buds in octyldodecyl myristate) from Gattefossé, ginger extracts, vitamin acetates, in particular vitamin E acetate, shea liquid and solid fractions, musk rose oil, Nutralipids® HY (mixture of passionflower, apricot kernel, maize and rice bran oil) from Nestlé, derivatives of these and mixtures of these. Mention may be made, as examples of fat-dispersible active agents suitable for the invention, of ellagic acid, glycyrrhetinic acid and their mixtures.

According to one embodiment, an oily phase of the invention can additionally comprise fat-soluble or fat-dispersible colouring agents, such as pigments or pearlescent agents.

Furthermore, active agents having a therapeutic effect used in the pharmaceutical field can also be encapsulated, in so far as they exhibit an oily nature and/or a satisfactory solubility and/or dispersibility in the oily phase under consideration.

They can be steroidal or nonsteroidal anti-inflammatories, antifungals, antibacterials, antibiotics, antimitotics, anaesthetics, analgesics, antiseptics or antivirals, indeed even mixtures of these.

Surface-Active Agents

According to one embodiment, a process according to the invention can employ at least one surface-active agent.

The surface-active agent(s) can be employed in the oily phase and/or in the aqueous phase.

A surface-active agent suitable for the invention may be selected from the group consisting in ionic, anionic, cationic or nonionic surface-active agents and their mixtures.

In order to facilitate the emulsification of the oily phase and, if appropriate, of the active agent to be encapsulated and to control the stability of the corresponding emulsion, it may be desirable to use at least one surface-active agent, in particular a nonionic surface-active agent.

A surface-active agent or a mixture of surface-active agents can be present at from 0.05 to 25% by weight and in particular from 1 to 20% by weight, with respect to the weight of the oily phase to be dispersed. Its HLB (Hydrophilic-Lipophilic Balance) value can be adjusted in order to be favourable to the formation of emulsions of oil-in-water type.

The following nonionic surface-active agents are suitable, for example, for the invention:
alkyl esters or ethers of glycerol or of polyglycerol composed of 1 to 10 glycerol “unit(s)” and of at least one alkyl chain (acid chain for the esters and alcohol chain for the ethers) having from 12 to 22 carbon atoms. It can be saturated or unsaturated and branched or unbranched. Mention may be made, as example, of Nikkol DGMS® (diglycerol monostearate), Nikkol decaglyn 21S® (decaglycerol diisostearate) or triglycerol hexadecyl ether,
mixed esters of fatty acids or of fatty alcohols, of carboxylic acid and of glycerol selected, for example, from the group consisting in mixed esters of C₈-C₂₂ fatty acid or fatty alcohol and of α-hydroxy acid and/or of succinic acid with glycerol and their mixtures. Mention may be made, as example, of the mixed ester of glycerol and of the mixture of citric acid, lactic acid, linoleic acid and oleic acid (CTFA name: Glyceryl citrate/lactate/linoleate/oleate) sold by Hills under the name Imwitor 375®; the mixed ester of succinic acid and of isostearyl alcohol with glycerol (CTFA name: Isostearyl diglyceryl succinate) sold by Hüls under the name Imwitor 780 K®; the mixed ester of citric acid and of stearic acid with glycerol (CTFA name: Glyceryl stearate citrate) sold by Hüls under the name Imwitor 370®; or the mixed ester of lactic acid and of stearic acid with glycerol (CTFA name: Glyceryl stearate lactate) sold by Danisco under the name Lactodan B30® or Rylo LA30®,
ethoxylated fatty ethers or ethoxylated fatty esters comprising from 2 to 50 ethylene oxide units and at least one alkyl chain (acid chain for the esters and alcohol chain for the ethers) having from 12 to 22 carbon atoms. The alkyl chain can be saturated or unsaturated and branched or unbranched. Mention will be made, as example, of the Brij® (ethoxylated fatty alcohols) series sold by Uniqema, the Myrj® (ethoxylated stearates) series sold by Uniqema and PEG 400 isostearate, also sold by Uniqema,
oxyethylenated or nonoxyethylenated sorbitan fatty esters. They comprise at least one sorbitan unit and at least one alkyl (fatty acid) chain having from 12 to 22 carbon atoms and, in the case where they are oxyethylenated, from 2 to 50 ethylene oxide units. The alkyl chain can be saturated or unsaturated and branched or unbranched. Mention will be made, as example, of the Span® (sorbitan esters) and Tween® (oxyethylenated sorbitan esters) series sold by Uniqema,
sugar fatty esters or sugar fatty ethers. The surfactant used is, for example, chosen from sucrose, maltose, glucose and fructose C₈ to C₂₂ fatty acid esters, methylglucose C₁₄ to C₂₂ fatty acid esters, alkylpolyglucosides and their mixtures. The alkyl chain or chains can be saturated or unsaturated and branched or unbranched.

The C₈-C₂₂ or C₁₄-C₂₂ fatty acids forming the fatty unit of the esters which can be used in the invention comprise at least one saturated or unsaturated linear alkyl chain respectively comprising from 8 to 22 or from 14 to 22 carbon atoms. The fatty unit of the esters can in particular be chosen from stearates, behenates, arachidonates, palmitates, myristates, laurates, caprates and their mixtures. Stearates can be used.

Mention may be made, as example of esters or of mixtures of esters of fatty acid and of sucrose, maltose, glucose or fructose, of sucrose monostearate, sucrose distearate, sucrose tristearate and their mixtures, such as the products sold by Croda under the name Crodesta® F50, F70, F110 and F160, respectively having an HLB (Hydrophilic-Lipophilic Balance) of 5, 7, 11 and 16, and, as example of esters or of mixtures of esters of fatty acid and of methylglucose, of polyglyceryl-3 methylglucose distearate, sold by Goldschmidt under the name Tegocare 450®. Mention may also be made of glucose or maltose monoesters, such as methyl O-hexadecanoyl-6-D-glucoside and such as O-hexadecanoyl-6-D-maltoside.

The ethers of fatty alcohol and of sugar which can be used as surfactants in the composition according to the invention can be chosen in particular from the group consisting of ethers and mixtures of ethers of C₈-C₂₂ fatty alcohol and of glucose, maltose, sucrose and fructose and ethers and mixtures of ethers of C₁₄-C₂₂ fatty alcohol and of methylglucose. These are in particular alkylpolyglucosides.

The C₈-C₂₂ or C₁₄-C₂₂ fatty alcohols forming the fatty unit of the ethers which can be used according to the invention comprise a saturated or unsaturated linear alkyl chain respectively comprising from 8 to 22 or from 14 to 22 carbon atoms. The fatty unit of the ethers can be chosen in particular from decyl, cetyl, behenyl, arachidyl, stearyl, palmityl, myristyl, lauryl, capryl, hexadecanoyl units and their mixtures, such as cetearyl.

Mention may be made, as example of ethers of fatty alcohol and of sugar, of alkylpolyglucosides, such as decyl glucoside and lauryl glucoside, sold, for example, by Henkel under the respective names Plantaren 2000® and Plantaren 1200®, cetearyl glucoside, optionally as a mixture with cetearyl alcohol, for example sold under the name Montanov 68® by Seppic, under the name Tegocare CG90® by Goldschmidt and under the name Emulgade KE3302® by Henkel, and arachidyl glucoside, for example in the form of the mixture of arachidyl and behenyl alcohols and of arachidyl glucoside sold under the name Montanov 202® by Seppic,
ethylene oxide and propylene oxide block copolymers. The ethylene oxide and propylene oxide block copolymers which can be used as surfactants in the compositions according to the invention can be chosen in particular from the block copolymers of formula (A):

HO(C₂H₄O)ₓ(C₃H₆O)_{y}(C₂H₄O)_{z}H (A)

in which x, y and z are integers such that x+z can range from 2 to 280 and can range from 14 to 100. These polymers are sold in particular under the Pluronic® or Lutrol® name by BASF or the Synperonic® name by Uniqema,
soybean or egg lecithins which are or are not hydrogenated and which are or are not enriched in phosphatidylcholine,
silicone surfactants comprising at least one oxyethylenated and/or oxypropylenated chain, Mention may be made, as example, of those described in U.S. Pat. Nos. 5,364,633 and 5,411,744 the content of which are incorporated by reference, for example a compound of formula (I):

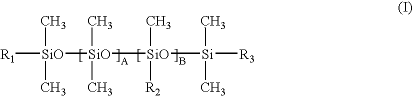

in which:

R₁, R₂ and R₃ represent, independently of one another, a C₁-C₆ alkyl radical or a —(CH₂)ₓ—(OCH₂CH₂)_{y}—(OCH₂CH₂CH₂)_{z}—OR₄ radical, at least one R₁, R₂ or R₃ radical not being an alkyl radical; R₄ being a hydrogen, an alkyl radical or an acyl radical;

A is an integer ranging from 0 to 200;

B is an integer ranging from 0 to 50; provided that A and B are not simultaneously equal to zero;

x is an integer ranging from 1 to 6;

y is an integer ranging from 1 to 30;

z is an integer ranging from 0 to 5.

According to a specific embodiment, in the compound of formula (I), the alkyl radical is a methyl radical, x is an integer ranging from 2 to 6 and y is an integer ranging from 4 to 30.

Mention may be made, as example of silicone surfactants of formula (I), of the compounds of formula (II):

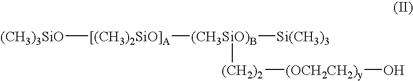

in which A is an integer ranging from 20 to 105, B is an integer ranging from 2 to 10 and y is an integer ranging from 10 to 20. Mention may also be made, as example of silicone surfactants of formula (I), of the compounds of formula (III):

HO—(CH₂CH₂O)_{y}—(CH₂)₃—[(CH₃)₂SiO]_{A}′—(CH₂)₃—(OCH₂CH₂)_{y}—OH (III)

in which A′ and y are integers ranging from 10 to 20.

Use may be made, as compounds, of those sold by Dow Corning under the names DC 5329, DC 7439-146, DC 2-5695 and Q4-3667. The compounds DC 5329, DC 7439-146 and DC 2-5695 are compounds of formula (II) where, respectively, A is 22, B is 2 and y is 12, A is 103, B is 10 and y is 12, and A is 27, B is 3 and y is 12. The compound Q4-3667 is a compound of formula (III) where A is 15 and y is 13. However, also:
alkyl ether citrates,
alkoxylated alkenyl succinates,
alkoxylated glucose alkenyl succinates,
alkoxylated methylglucose alkenyl succinates.

These surfactants can be used alone or in combination. Their level, with respect to the encapsulated oily phase, can be between 0.1 and 30% by weight.

It may be possible, in order to improve the stability of the emulsion, indeed even to reduce somewhat further the size of the drops, to add from 0.01 to 5% by weight, with respect to the total weight of the dispersion, of at least one water-soluble ionic surfactant having an HLB of greater than 11. This type of ionic surfactant appears to generate an electric charge at the surface of the nanocapsules and thus promotes the appearance of electrostatic repulsions between them. This or these anionic or cationic ionic surfactants can be chosen from:
anionic amphiphilic lipids, such as:
   the alkaline salts of dicetyl and dimyristyl phosphate;
   the alkaline salts of cholesterol sulphate;
   the alkaline salts of cholesterol phosphate;
   lipoamino acids and their salts, such as mono- and disodium acylglutamates, such as the disodium salt of N-stearoyl-L-glutamic acid sold under the name Amisoft HS21P by Ajinomoto;
   the sodium salts of phosphatidic acid;
   phospholipids;
   alkylsulphonic derivatives, in particular of formula:

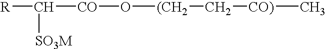

in which R represents C₁₆-C₂₂ alkyl radicals, in particular the C₁₆H₃₃ and C₁₈H₃₇ radicals, taken as a mixture or separately, and M is an alkali metal or alkaline earth metal, such as sodium.
cationic amphiphilic lipids of the following types: quaternary ammonium salts, fatty amines and their salts, such as, for example:
   quaternary ammonium salts of following general formula (IV):

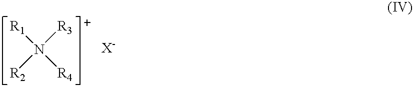

in which the R₁, R₂, R₃ and R₄ radicals, which can be identical or different, represent a linear or branched aliphatic radical comprising from 1 to 30 carbon atoms or an aromatic radical, such as aryl or alkylaryl. The aliphatic radicals can comprise heteroatoms, such as, in particular, oxygen, nitrogen, sulphur or halogens. The aliphatic radicals are, for example, chosen from alkyl, alkoxy, polyoxy(C₂-C₆)alkylene, alkylamide, (C₁₂-C₂₂)alkylamido(C₂-C₆)alkyl, (C₁₂-C₂₂)alkyl acetate or hydroxyalkyl radicals comprising approximately from 1 to 30 carbon atoms; X is an anion chosen from the group consisting of halides, phosphates, acetates, lactates, (C₂-C₆)alkyl sulphates, and alkyl- and alkylarylsulphonates. Preference is given, as quaternary ammonium salts of formula (IV), to, on the one hand, tetraalkylammonium chlorides, such as, for example, dialkyldimethylammonium or alkyltrimethylammonium chlorides in which the alkyl radical comprises approximately from 12 to 22 carbon atoms, in particular behenyltrimethylammonium, distearyldimethylammonium, cetyltrimethylammonium and benzyldimethylstearylammonium chlorides, or alternatively, on the other hand, stearamidopropyldimethyl(myristyl acetate)ammonium chloride, sold under the name ACeraphyl 70®@ by Van Dyk,
imidazolinium quaternary ammonium salts, such as, for example, those of following formula (V):

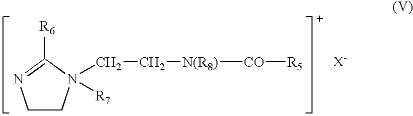

in which R₅ represents an alkenyl or alkyl radical comprising from 8 to 30 carbon atoms, for example derived from tallow fatty acids; R₆ represents a hydrogen atom, an alkyl radical comprising from 1 to 4 carbon atoms or an alkenyl or alkyl radical comprising from 8 to 30 carbon atoms; R₇ represents an alkyl radical comprising from 1 to 4 carbon atoms; R₈ represents a hydrogen atom or an alkyl radical comprising from 1 to 4 carbon atoms; X is an anion chosen from the group consisting of halides, phosphates, acetates, lactates, alkyl sulphates, and alkyl- and alkylarylsulphonates. R₅ and R₆ can denote a mixture of alkenyl or alkyl radicals comprising from 12 to 21 carbon atoms, for example derived from tallow fatty acids, R₇ denotes a methyl radical and R₈ denotes hydrogen. Such a product is, for example, sold under the name ARewoquat W 75@ by Rewo,
diquaternary ammonium salts of following formula (VI):

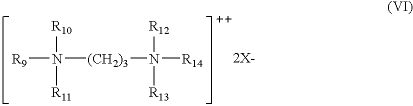

in which R₉ denotes an aliphatic radical comprising approximately from 16 to 30 carbon atoms; R₁₀, R₁₁, R₁₂, R₁₃ and R₁₄ are chosen from hydrogen or an alkyl radical comprising from 1 to 4 carbon atoms; and X is an anion chosen from the group consisting of halides, acetates, phosphates, nitrates and methyl sulphates. Such diquaternary ammonium salts comprise in particular propanetallowediammonium dichloride.

The level of ionic surfactant, when it is combined with the nonionic surfactant(s) present in the aqueous medium, can be adjusted so as to represent from 2 to 100% by weight of the weight of the latter.

Compounds Capable of Forming a Lamellar Phase

It is often desirable or necessary to provide the capsules of the invention with a “lamellar” coating. It is a structure organized as one or more lipid bilayer(s) each composed of a bilayer of amphiphilic molecules which are similar to that of biological membranes.

The polymeric casing of the capsules according to the invention can thus be surrounded with a lamellar coating having a structure organized as one or more bilayer(s) each composed of a double layer of amphiphilic molecules constituting a coating agent. This coating, in addition to its role of adjusting the size of the capsules, improves the leaktightness of the capsules with regard to leakage of active principle towards another lipid phase of the composition.

The term “lamellar phase” (phase D according to Ekwall) is understood to mean a liquid crystal phase with plane symmetry comprising several amphiphilic double layers arranged in parallel and separated by a liquid medium which is generally water.

A more precise definition of this name is given in Ekwall (1968), Adv. Liq. Cryst. (edited by C. H. Brown), Chap. 1, 14, the content of which being incorporated herein by reference, This phase has a characteristic texture under a polarized light microscope, a more detailed description of which can be found in Roservear (1968), JSCC, 19, 581, and in Lachampt and Vila (1969), Revue Française des Corps Gras, No. 2, 87-111, the content of which being incorporated herein by reference.

The lamellar coating is obtained with surface-active agents having a hydrophobic nature which are soluble in the oily phase used in the process described above and which are capable, in the presence of water, of forming the lipid double layers described above. In the encapsulation process used by the inventors, the coating surfactant can be dissolved in the oily phase comprising oligomers and, if appropriate, the active agent(s).

Mention may be made, as example of such coating surfactants, of phospholipids, such as lecithin, as described in the document EP-A-447 318 (or U.S. Pat. No. 6,203,802), the content of which being incorporated herein by reference; certain ethylene oxide and propylene oxide polycondensates, such as the products sold under the Pluronic® name by BASF, such as Pluronic® L121, or under the Synperonic® name by ICI; or silicone surface-active agents (silicones comprising at least one oxyethylenated and/or oxypropylenated chain) capable of forming lamellar structures, such as those described in the documents U.S. Pat. No. 5,364,633 and U.S. Pat. No. 5,411,744 the content of which being incorporated herein by reference used in Patent Application FR-A-2 742 677 (or U.S. Pat. No. 5,919,487) the content of which being incorporated herein by reference, for example those sold by Dow Corning under the names DC 5329, DC 7439-146, DC 2-5695 and Q4-3667; and their mixtures.

Crosslinking Agents

According to one embodiment, a process in accordance with the invention may comprise a crosslinking step g). This crosslinking step can be carried out in the presence of at least one crosslinking agent.

A crosslinking step is advantageously carried out when the melting point Mp of the polymer or polymers, in particular of the oligomers, employed is less than ambient temperature T_{amb}.

Mention may be made, as examples of crosslinking agents suitable for the invention, of molecules which are difunctional at least or polyfunctional.

The crosslinking agent(s) can be of hydrophilic nature for incorporation in the aqueous phase.

The crosslinking agent(s) can be of lipophilic nature for incorporation in the oily phase.

In order in particular to prevent a detrimental reaction between the encapsulated active agent(s) and the crosslinking agent, the latter will be chosen in particular from hydrophilic crosslinking agent(s).

Such an agent will in particular be added at the end of the process via the aqueous phase.

Mention may be made, as examples of identical or different functional groups carried by a crosslinking agent suitable for the invention, of isocyanate, mixed anhydride and acid chloride.

A crosslinking agent suitable for the invention can be chosen in particular from polyisocyanates.

The polyisocyanates can be hexamethylene diisocyanate (HDI) oligomers (such as the Rhodocoat® products from Rhodia and the Basonat® products from BASF), toluene diisocyanate (TDI), such as Vibrathane 6060 from Uniroyal Chemical, methylenebis(phenylisocyanate) (MDI), such as Vibrathane 8030 and Vibrathane 8045, or isophorone (IPDI), such as the Hypol products from Dow Chemicals.

The hydrophilic crosslinking agents can be chosen from Rhodocoat WT2102 from Rhodia, Basonat HW100 from Bayer, Bayhydur from Bayer or Hypol JT from Dow Chemicals.

The lipophilic crosslinking agents can be chosen from Rhodocoat HDT-LV from Rhodia or Desmodur from Bayer.

Cosmetic, Dermatological or Pharmaceutical Composition

The capsules of the invention can be introduced into any type of pharmaceutical formulation, such as gels, oil/water, water/oil or multiple (W/O/W, O/W/O) emulsions, serums, lotions and the like.

According to one embodiment, a suspension of capsules of the invention can be employed in a composition in a content varying from 0.5 to 60% by weight, with respect to the total weight of the composition, in particular varying from 0.5 to 40% by weight and more particularly varying from 2 to 10% by weight, with respect to the total weight of the composition.

A suspension of capsules of the invention can be used for the preparation of a cosmetic, dermatological or pharmaceutical composition.

Such compositions can in particular be intended to convey, in particular in improved fashion, cosmetic or therapeutic active principles. Thus, these compositions can confer an improved bioavailability on these active principles.

Thus, a subject-matter of the invention is a cosmetic, dermatological or pharmaceutical composition comprising, in a physiologically acceptable medium, at least one aqueous suspension of capsules which is capable of being obtained according to a process of the invention.

The formulations comprising capsules can, for example, be intended for caring for and/or making up the hair, skin or nails.

Furthermore, the capsules of the present invention can be used for pharmaceutical purposes for the vectorization of medicaments via the oral, peritoneal, intravenous, intramuscular or ophthalmic route.

In addition, the capsules of the invention can be used for the preparation of cosmetic compositions which can be provided in the form of a mascara, product for the eyebrows, eyeliner, eye shadow, blusher, foundation, product for the lips, product for making up the body or product for making up or caring for the hair.

The cosmetic, dermatological or pharmaceutical compositions of the invention can comprise any additive normally used in the fields concerned, with the proviso that these additives do not detrimentally affect the property of the compositions or of the capsules of the invention.

The examples, given below purely by way of illustration and without implied limitation, will make possible a better understanding of the invention.

The amounts are indicated therein as percentage by weight, unless otherwise indicated.

### EXAMPLE 1

### Shea Butter Capsules

Organic phase:

| | | |
|---|---|---|
| Polycaprolactone oligomer Capa ® 2200A from | 2 | g |
| SOLVAY | | |
| Shea butter (Lipex 202 from Karlshamns) | 7 | g |
| Isopropyl N-lauroylsarcosinate (Eldew SL-205 from | 8 | g |
| Ajinomoto) | | |

Aqueous phase:

| | | |
|---|---|---|
| Disodium N-stearoyl-L-glutamate (Amisoft HS21P | 0.5 | g |
| from Ajinomoto) | | |
| Distilled water | q.s. for 100 | g |

The oily and aqueous phases are heated to 80° C. An organic/aqueous preemulsion is prepared using an UltraTurrax stirrer (Ika) at 10 000 rpm for 5 min in a bath thermostatically controlled at 80° C. This preemulsion is subsequently homogenized, at 80° C., 2 times at 600 bar, using a high pressure homogenizer of OBL20 type from Niro Soavi.

Subsequently, the emulsion is cooled to ambient temperature by simple stirring with a magnetic bar.

A suspension of capsules comprising 7% of shea butter and 2% of oligomer by weight is obtained.

The size of the capsules is monitored using a laser particle sizer by light scattering, as indicated above. The mean size of the capsules is 170 nm.

The capsules obtained have a polydispersity index, measured as indicated above, of 0.183.

These capsules are perfectly stable for 2 months at +4° C., at ambient temperature and at 45° C.

### EXAMPLE 2

### Postcrosslinking of the Shea Capsules

The shea capsules are prepared according to Example 1.

| | | |
|---|---|---|
| Shea capsules | 100 | g |
| Bayhydur 3100 (Bayer) | 0.5 | g |

0.5 g of Bayhydur 3100 is added to 100 g of suspension of capsules with stirring at ambient temperature in a fumed cupboard.

### EXAMPLES 3-19

Capsules with (Examples 3-11) or without (Examples 12-17) oily active agent are prepared as indicated in Example 1.

The formulation of these compositions also comprises 0.5% of acylglutamate HS 21 from Ajinomoto and distilled water, q.s. for 100.

The results obtained are summarized in the table below.

| | | | | Formation of capsules |
|---|---|---|---|---|
| | Name of the active | Associated oils | | Mean diameter |
| Ex. | principle and % | and % | Oligomers | Polydispersity index |
| 3 | Gatuline Derma Sensitive | Eldew SL 205 | Capa ® 2302A | OK |
| | 4.5% | 10.5% | 2% | 172 nm |
| | | | | 0.113 |
| 4 | Ginger extract | Capric/caprylic acid | Capa ® 2200A | OK |
| | 3.75% | triglycerides | 2% | 173 nm |
| | | 11.25% | | 0.193 |
| 5 | Ginger extract | Capric/caprylic acid | Capa ® 2302A | OK |
| | 3.75% | triglycerides | 2% | 161 nm |
| | | 11.25% | | 0.091 |
| 6 | Vitamin E acetate | Capric/caprylic acid | Capa ® 3050 | OK |
| | 0.66% | triglycerides | 0.5% | 166 nm |
| | | 12.6% | | 0.137 |
| 7 | Vitamin E acetate | Capric/caprylic acid | Capa ® 2200A | OK |
| | 18.25% | triglycerides | 2.5% | 320 nm |
| | | 0.75% | | 0.250 |
| 8 | Vitamin E acetate | Capric/caprylic acid | Capa ® 2200A | OK |
| | 18.25% | triglycerides | 2.5% + | 286 nm |
| | | 0.75% | Capa ® 3050 | 0.255 |
| | | | 0.5% | |
| 9 | Shea liquid fraction | Eldew SL 205 | Capa ® 2200A | OK |
| | 7% | 8% | 2% | 160 nm |
| | | | | 0.183 |
| 10 | Nutralipids HY | Eldew SL 205 | Capa ® 2200A | OK |
| | 7.5% | 7.5% | 2% | 179 nm |
| | | | | 0.096 |
| 11 | Musk rose oil | Eldew SL 205 | Capa ® 2200A | OK |
| | 9.4% | 5.6% | 2% | 172 nm |
| | | | | 0.146 |
| 12 | Shea liquid fraction | Eldew SL 205 | Capa ® 2200A | OK |
| | 3.3% + | 8.4% | 2% | 192 nm |
| | shea solid fraction | | | 0.139 |
| | 3.3% | | | |
| 13 | | Isocetyl stearate | Eternacoll 3020 | OK |
| | | 8.6% + | (UBE) | 180 nm |
| | | Eldew SL 205 | 2% | 0.140 |
| | | 6.4% | | |
| 14 | | Isocetyl stearate | UH-Carb 50 (UBE) | OK |
| | | 2% + | 2% | 160 nm |
| | | Capric/caprylic acid | | 0.187 |
| | | triglycerides | | |
| | | 13% | | |
| 15 | | Capric/caprylic acid | UH-Carb 300 (UBE) | OK |
| | | triglycerides | 2% | 192 nm |
| | | 10% + | | 0.150 |
| | | Eldew SL 205 | | |
| | | 5% | | |
| 16 | | Capric/caprylic acid | Fomrez F930 | OK |
| | | triglycerides | (Crompton) | 165 nm |
| | | 5.6% + | 2% | 0.157 |
| | | Eldew SL 205 | | |
| | | 9.4% | | |
| 17 | | Capric/caprylic acid | Lexorez 1151-35 | OK |
| | | triglycerides | (Inolex) | 167 nm |
| | | 3.75% + | 2% | 0.147 |
| | | Eldew SL 205 | | |
| | | 11.25% | | |
| 18 | | Capric/caprylic acid | Lexorez 1460-36 | OK |
| | | triglycerides | (Inolex) | 182 nm |
| | | 8.75% + | 2% | 0.128 |
| | | Eldew SL 205 | | |
| | | 6.25% | | |
| 19 | Liquid fraction of shea | Eldew SL 205 | Capa ® 2201 | OK |
| | butter | 11.2% | (Solvay) | 220 nm |
| | 4.4% | | | 0.141 |
| | Solid fraction of shea | | | |
| | butter | | | |
| | 4.4% | | | |

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Process for the preparation of a suspension of capsules of core/shell type comprising at least the steps of:
a) having a heterogeneous mixture at ambient temperature (T_{amb}) comprising:
an oily phase,
identical or different polymers which are immiscible with water and with the oily phase, at least one polymer of which is an oligomer having a weight-average molecular weight of less than 10 000 and a hydroxyl number (N_{OH}) of greater than or equal to 10 mg KOH/g,
b) homogenizing the oily phase and the polymers by heating the mixture obtained in step a) at a homogenization temperature T_{H}, T_{H} being less than 100° C., the said mixture having, at the temperature T_{H}, a viscosity of less than or equal to 17 mPa·s,
c) dispersing, at the temperature T_{H}, the homogeneous mixture obtained in step b) in an aqueous phase, in order to obtain a direct preemulsion, the oily phase of which comprises the polymers,
d) subjecting the preemulsion obtained in step c) to a combination of shear forces appropriate for the reduction in the diameter of the particles of the dispersed mixture to a mean size of less than or equal to approximately 50 μm,
e) cooling the emulsion obtained in step d) to a temperature T_{c} suitable for the coacervation of the said polymers and for the coating of drops of the said oily phase by the coacervates, and
f) cooling the suspension obtained in step e) to a temperature Tₚ suitable for the formation of the expected capsules by the precipitation and/or crystallization of the coacervates,
the oily phase being suitable for the formation of a homogeneous mixture with the said polymers, at a temperature T_{H}, and for the appearance of a phenomenon of coacervation of the said polymers at a temperature T_{c}.

2. Process according to claim 1, comprising a step g) of crosslinking of the polymers.

3. Process according to claim 1, in which T_{c} is greater than or equal to 45° C.

4. Process according to claim 1, in which the said homogeneous mixture has, at the temperature T_{H}, a viscosity varying from 1 to 17 mPa·s.

5. Process according claim 1, in which the capsules have a mean diameter in size varying from 50 nm to 10 μm.

6. Process according claim 1, in which the suspension of capsules exhibits a level of capsules varying from 3 to 90% by weight, with respect to the total weight of the suspension.

7. Process according claim 1, in which the polymers and the oily phase are present according to a ratio by weight varying from 5/1 to 1/20.

8. Process according claim 1, in which the oily phase comprises at least one fat-soluble active agent and/or one fat-dispersible active agent.

9. Process according claim 8, in which the oily phase and the active agent(s) are present according to a ratio by weight varying from 100/0 to 1/99.

10. Process according claim 1, in which the said oligomer(s) comprise at most 100 monomer units.

11. Process according claim 1, in which the said oligomer(s) have a weight-average molecular weight of greater than or equal to 400 and less than 1 0 000.

12. Process according claim 1, in which the said oligomer(s) have a hydroxyl number (N_{OH}) at least equal to 10 mg KOH.

13. Process according claim 1, comprising, in step a), polymers with a molecular weight of greater than or equal to 10 000.

14. Process according claim 1, in which the said polymers have a melting point Mp of less than 100° C.

15. Process according claim 1, in which the said oligomer(s) are selected from the group consisting in polycaprolactones, poly-L-lactides, poly-DL-lactides, polyglycolides and their copolymers, polymers of 3-hydroxybutyric acid and its copolymers with hydroxyvaleric acid, polycarbonate diols, polyalkylene adipates, polyester polyols, dendritic polyesters comprising an end hydroxyl functional group, and their blends.

16. Process according claim 1, in which the oily phase forms an active agent.

17. Process according claim 1, in which the oily phase comprises at least one oil selected from the group consisting in nonvolatile oils, and mixtures of these.

18. Process according claim 17, in which the oily phase comprises at least one oil selected from the group consisting in hydrocarbon oils of animal origin, hydrocarbon oils of vegetable origin, linear or branched hydrocarbons of mineral or synthetic origin, synthetic ethers having from 10 to 40 carbon atoms, synthetic esters of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂≧10, non volatile silicone oils, and their mixtures.

19. Process according to claim 8, in which the said active agent is a fat-soluble active agent selected from the group consisting in natural, vegetable, animal or synthetic oily substances which are liquid from 40° C., vegetable oils rich in unsaturations, sunscreens, vitamins E, F and K, their esters and their mixtures, vitamins, carotenes, salicylic acid, ceramides, α-linoleic acid, Gatuline Derma Sensitive® (extract of caper buds in octyldodecyl myristate), ginger extracts, shea liquid and solid fractions, musk rose oil, Nutralipids® (mixture of passionflower, apricot kernel, maize and rice bran oil) and derivatives of these, steroidal or nonsteroidal anti-inflammatories, antifungals, antibacterials, antibiotics, antimitotics, anaesthetics, analgesics, antiseptics, antivirals and their mixtures.

20. Process according to claim 8, in which the said active agent is a fat-dispersible active agent selected from the group consisting in ellagic acid, glycyrrhetinic acid and their mixtures.

21. Process according to claim 1, in which at least one surface-active agent is employed in the oily phase and/or in the aqueous phase.

22. Process according to claim 21, in which the surface-active agent is selected from the group consisting in ionic, anionic, cationic or nonionic surface-active agents and their mixtures.

23. Process according to claim 2, in which the crosslinking step g) employs at least one crosslinking agent selected from the group consisting in hydrophilic or lipophilic molecules which are difunctional or polyfunctional.

24. Process according to claim 23, in which the identical or different functional groups of the crosslinking agent are selected from the group consisting in isocyanate, mixed anhydride or acid chloride.

25. Process according to claim 23, in which the crosslinking agent is a polyisocyanate selected from the group consisting in hexamethylene diisocyanate oligomers, toluene diisocyanate or methylenebis(phenyl isocyanate).

26. Process for the preparation of a cosmetic, dermatological or pharmaceutical composition comprising at least the stage of mixing, with a physiologically acceptable medium, a suspension of capsules which is obtained according to the process as defined according to claim 1.

27. Cosmetic, dermatological or pharmaceutical composition comprising, in a physiologically acceptable medium, a suspension of capsules which is capable of being obtained according to the process as defined according to claim 1.

28. Composition according to claim 27, in which the suspension of capsules is present in a content varying from 0.5 to 60% by weight with respect to the total weight of the composition.

29. Capsules capable of being obtained by means of the process as defined according to claim 1.

30. Aqueous suspension of capsules which is capable of being obtained by means of the process as defined according to claim 1.
